(19) United States
(12) Patent Application Publication
(10) Pub. No.: US 2016/0106359 A1
(43) Pub. Date: Apr. 21, 2016

(54) SPHINCTER TEST CATHETER

(71) Applicant: UNISENSOR AG, Attikon (CH)

(72) Inventor: Peter SCHLUMPF, Ellikon an der Thur (CH)

(21) Appl. No.: 14/890,458

(22) PCT Filed: May 28, 2014
(86) PCT No.: PCT/EP2014/061142

§ 371 Date: Nov. 11, 2015

(30) Foreign Application Priority Data
May 30, 2013 (CH) 1038/13
May 27, 2014 (CH) 807/14

(51) Int. Cl. A61B 5/00; A61B 5/11; A61B 5/053

(52) U.S. Cl. CPC A61B 5/4222; A61B 5/0538; A61B 5/1116

(57) ABSTRACT

The sphincter test catheter includes a catheter body, the proximal end of which forms a probe. Corresponding electric lines which are led to a recording unit run through the catheter body. The sphincter test catheter has at least one pressure sensor and a position sensor in combination with either a pH value sensor or an impedance sensor. The combination depends on which sphincter is to be monitored. The position sensor is combined with a pH value sensor in order to monitor the lower esophageal sphincter while the position sensor is combined with an impedance sensor when monitoring the urethral sphincter.

The present invention relates to a sphincter test catheter for testing and monitoring the function of an intracorporeal sphincter, comprising a flexible catheter body having a probe arranged at the proximal end thereof, in which probe at least one pressure sensor is provided, wherein the sphincter test catheter is connected to a measuring and recording unit.

Sphincters are annular constricting muscles that are intended to be able to close off a muscular hollow organ. Such annular constricting muscles are present extracorporeally and intracorporeally. In the present invention, however, only the intracorporeal sphincters are of interest, in this case in particular the urethral sphincter, which closes off the bladder from the urethra, and the lower oesophageal sphincter, which is also known as the cardiac sphincter and in terms of function is indeed a sphincter, but is not formed by an annular constricting muscle, but instead by a narrowing loop of the diaphragm.

Sphincter test catheters are known in various embodiments and have different sensors in the region of the probe depending on the sphincter to be tested. Here, these test catheters are used to show snapshots, which are useful for the diagnosis. If the closing force of the urethral sphincter is to be tested, a catheter is introduced through the urethra, by means of which catheter water is conveyed into the bladder and the pressure in the bladder is measured continuously whilst the pressure in the bladder increases due to the feed of water until a moment at which a further sensor, for example an impedance sensor, determines that urine has escaped from the bladder. The function of the oddi sphincter can also be tested in a similar manner. A testing of the lower oesophageal sphincter was not previously measured, but instead was usually only examined optically by means of an introduced catheter having an image sensor and optical waveguide. Since, until now, all sphincter test catheters are used only to determine a momentary value, the examining doctor of course also knew the position of the patient during this test.

Different examinations that are carried out over a longer period of time are highly dependent, in terms of the measured values, on the position currently assumed by a patient. Such tests are performed in particular in what are known as sleep laboratories. Here, a relationship between different bodily functions and the movements and the position of a patient as he/she sleeps is to be determined. Here, however, there is no monitoring of intracorporeal sphincter functions.

Since, in a sleep laboratory, the patient is not at home and is continuously monitored by medically trained staff, the patient is provided here with a very large number of different sensors and conductors, which are all guided separately to corresponding recording and measuring apparatuses.

The object of the present invention is now to create a sphincter test catheter that is suitable for testing and monitoring an intracorporeal sphincter over a relatively long period of time.

This object is achieved by a sphincter test catheter of the type mentioned in the introduction, which has the characterising features of claim 1.

For the functions of a sphincter that are to be monitored here, a orientation sensor is first installed in a sphincter test catheter in accordance with this concept. The spatial orientation of the test catheter can be determined using the orientation sensor, which is also referred to as an XYZ sensor or gyroscope sensor.

In the event of long-term testing, as is performed in sleep laboratories, orientation sensors are indeed also used, but are arranged extracorporeally. This would also be implemented in an obvious manner in the present case, however it has been found that it is much more comfortable for the patient to be connected merely to one measuring probe than to a plurality of probes having corresponding connection cables. In addition an intracorporeal exact position determination of certain sensors can be attained with the present solution.

A further advantage of the present solution lies in the fact that when testing the lower oesophageal sphincter over a longer time a possible gastro-oesophageal reflux can be examined depending on the inclination or change in inclination of the oesophagus, for example during sleep or when eating.

Furthermore, the test catheter can be used with a orientation sensor for examining the spatial course of the oesophagus or the urethra, for example by being removed from the patient at a constant speed (typically in the range of 1-50 mm/minute) using a catheter pulling device (what is known as a catheter puller). For this purpose the measuring and recording unit is configured in such a way that it determines, and records, the spatial orientation of the orientation sensor or the change in orientation in accordance with the time over which the catheter is removed, for example from the oesophagus or urethra. In this way, it is possible to determine a prolapsed bladder, for example in female patients. Here, the measuring and recording unit calculates the spatial course of the oesophagus or urethra from the time-dependent change of the spatial orientation of the orientation sensor.

By removing the catheter at constant speed with the pressure sensor, the length of the active sphincter can also be determined in that the measuring and recording unit is configured accordingly.

It is well known from the prior art to also provide catheters with a orientation sensor, however this serves for a completely different purpose. Catheters for cardiology are thus provided with orientation sensors in order to be able to determine the spatial position of the tip in the patient in order to facilitate the guidance of the catheter for surgeons. Here, this therefore in no way involves a monitoring of the physical position of a patient, i.e. does not involve a monitoring as to whether the patient is lying down, standing up or sitting, since the patient is always under the control of the cardiologist in the case of such interventions. Examples of such catheters are known from EP 1240868 or US 2013/0096551. A combination with other sensors for determining pH values or for determining the impedance is not known in this context and also is not useful.

The present invention, however, as the closest prior art, proceeds from a sphincter test catheter that is suitable for monitoring the function of an intracorporeal sphincter and has a flexible catheter body in the proximal end of which a probe is arranged, said probe having at least one pressure sensor. For monitoring and recording, this sphincter test catheter additionally can be brought into connection with a measuring and recording unit.

Two different preferred exemplary embodiments of the subject matter of the invention are illustrated in the drawing and will be explained in the following description.

In the drawing:

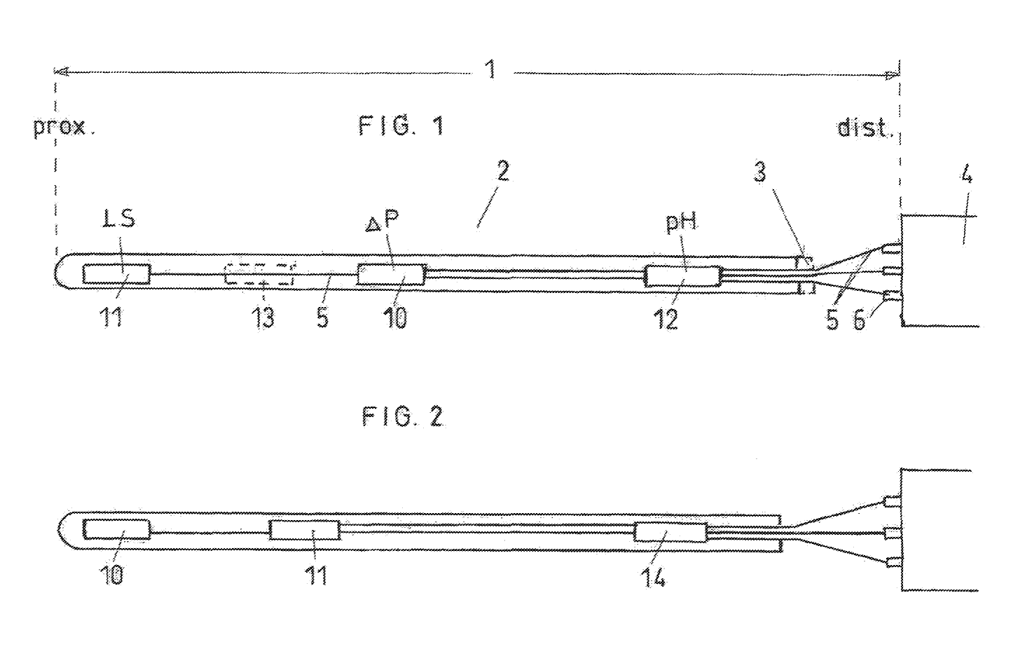

FIG. 1 shows a schematic illustration of a sphincter test catheter for testing and monitoring the lower oesophageal sphincter, and

FIG. 2 likewise shows a schematic illustration of a test catheter for testing and monitoring the urethral sphincter.

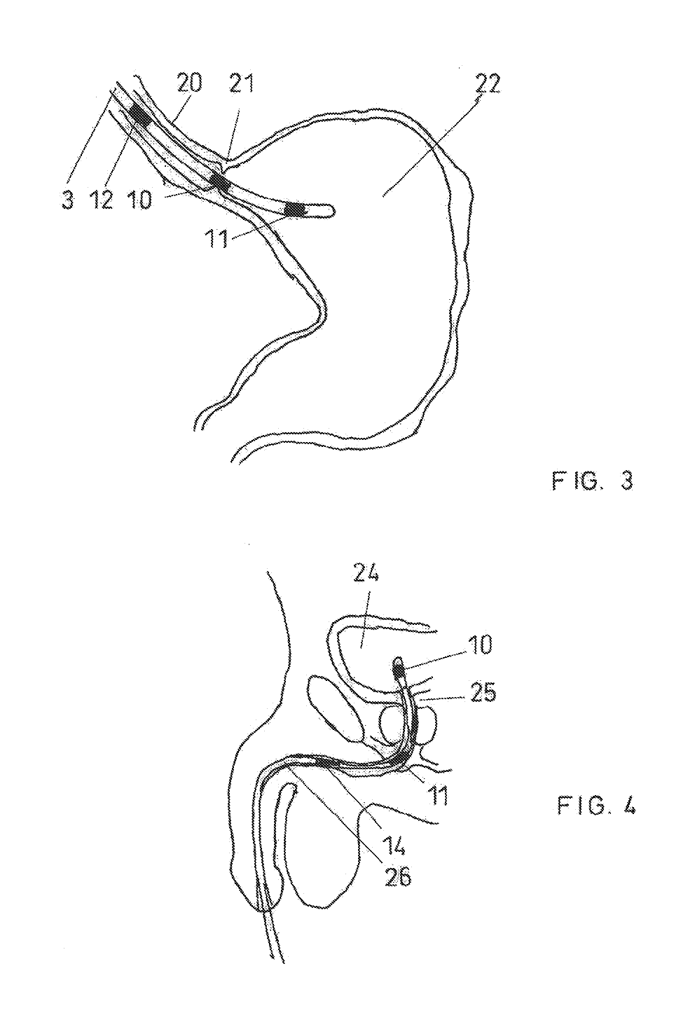

FIG. 3 schematically shows the catheter introduced into a stomach for monitoring the function of a lower oesophageal sphincter;

FIG. 4 shows a catheter inserted in a urethra for testing and monitoring the male urethral sphincter, and

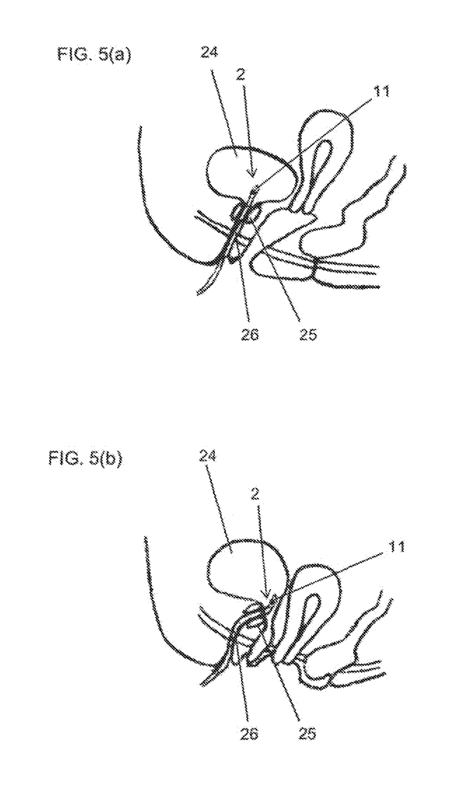

FIG. 5 shows a catheter inserted in a urethra for testing and monitoring the female urethral sphincter (with (a) and without (b) prolapsed bladder).

FIG. 1 shows a sphincter test catheter for testing and monitoring the lower oesophageal sphincter. Reference sign 1 designates the entire catheter, which in principle leads from the tip of the probe, i.e. from the proximal end of the catheter, to the recording unit 4. The end that leads to the recording unit 4 is the distal end of the sphincter test catheter. The proximal end region of the sphincter test catheter 1, in which the different sensors are arranged, is designated as the probe 2. In contrast to other known catheters, this probe is not specially shaped, but here constitutes simply the proximal end region of the catheter body 3. The catheter body 3 thus consists merely of a protective sleeve in which the necessary electrical leads extend. The ends of these electrical conductors 5 are provided with corresponding sockets or clamping sleeves 6 in order to produce the electrical and mechanical connection to the recording unit 4. This recording unit 4 will be discussed in greater detail further below.

The arrangement and order of the different sensors within the probe 2 is significant and is adapted to the use, depending on whether the sphincter test catheter 1 is a catheter for testing the lower oesophageal sphincter or a sphincter test catheter for testing the urethral sphincter.

The version according to FIG. 1 will be discussed first and constitutes a sphincter test catheter for monitoring the lower oesophageal sphincter. This sphincter test catheter has, at its proximal end, a orientation sensor 11, which is followed in the distal direction by a pressure sensor 10. This pressure sensor 10 is then followed by a pH measurement value sensor 12. This pH measurement value sensor is then followed by the flexible catheter body 3, of which the length is actually a multiple of the length of the probe 2. It may possibly also be beneficial to monitor the pH value of the stomach contents. In that case, an additional pH value sensor 13 is arranged between the proximal orientation sensor 11 and the pressure sensor 10, as illustrated by dashed lines.

The sphincter test catheter 1 according to the variant in FIG. 2 is particularly suitable for monitoring the urethral sphincter. Here, a pressure sensor 10 is arranged in the proximal end of the probe 2. This pressure sensor 10 is then followed in the distal direction by a orientation sensor 11. The orientation sensor 11 is then followed likewise in the distal direction by an impedance sensor 14.

Since, as mentioned, the sphincter test catheter is not used to measure momentary values, and accordingly no state-changing interventions are necessary, such as the feeding of a saline solution in order to increase the pressure in the bladder, such sphincter test catheters according to the invention are extraordinarily thin. Merely the sensors and the electrical connections, i.e. the electrical conductors 5, must have space besides the sensors in the probe or on the catheter. This leads to a catheter having a very small diameter of approximately 2-4 mm in cross section. The thinner are such sphincter test catheters, the less irritation these cause in the patient when such a catheter is carried in the patient for example for 24 hours.

FIG. 3 now shows a sphincter test catheter according to FIG. 1 in the use position. The lower oesophageal sphincter 21 can be seen between the stomach 22 and the oesophagus 20. The orientation sensor 11 protrudes into the stomach 22 and rests for example on the smaller stomach arc. The pressure sensor 10 is placed in the region of the lower oesophageal sphincter 21, and the pH measurement value sensor 12 now lies accordingly in the region of the oesophagus 20 or distally of the lower oesophageal sphincter 21. Thanks to this arrangement of the sensors within this sphincter test catheter, the sphincter test catheter can be placed extraordinarily accurately. As soon as the pressure sensor indicates the maximum value, it is located in the region of the lower oesophageal sphincter. Of course, the passage through the upper oesophagus is also determined with the pressure sensor, however this is of no significance here.

If, when monitoring the function of the lower oesophageal sphincter 21, the pH value of the stomach should also be checked at the same time, an additional pH value sensor can thus be arranged without difficulty between the orientation sensor 11 and the pressure sensor 10. In principle, this additional pH value sensor 13 could also be arranged in front of the orientation sensor 11 in the proximal direction. In this case, this additional pH value sensor would be located permanently in the gastric juice, whereas, with the arrangement of the additional pH value sensor 13 between the orientation sensor 11 and the pressure sensor 10, said sensor is located at the entry to the stomach. This additional pH value sensor thus lies within the gastric juice only in certain body positions and/or only when the stomach is full to a certain level. Accordingly, the additional pH value sensor, when desired, is arranged in one or other of the previously described positions.

Should the inclination of the proximal portion of the oesophagus 20 also be determined when monitoring the function of the lower oesophageal sphincter 21, the orientation sensor 11 may also be arranged after the pressure sensor 10 in the distal direction, such that said orientation sensor comes to lie in the proximal portion of the oesophagus 20 once the sphincter test catheter 1 has been inserted.

A sphincter test catheter 1 for monitoring the function of the lower oesophageal sphincter 21 may additionally have an impedance sensor after the pressure sensor 10 in the distal direction, in which impedance sensor the individual measuring rings extend over 5 to 20 cm along the catheter, for example in order to determine the strength of a gastro-oesophageal reflux or the propagation thereof along the oesophagus. The distance between adjacent rings is generally 0.5 to 2.5 cm.

FIG. 4 shows a sphincter test catheter 1 according to the embodiment in FIG. 2 when used to monitor the urethral sphincter. The sphincter test catheter 1 is advanced here until the pressure sensor 10 lies in the bladder 24. The orientation sensor 11 lies in the urethra 26 after the urethral sphincter 25 in the distal direction. Here, the orientation sensor 11 is in a stable position. The orientation sensor 11 is then followed in the distal direction by the impedance sensor 14 in the urethra 26.

FIG. 5 shows a sphincter test catheter 1 different from FIG. 4 in the use for monitoring the female urethral sphincter. If the sphincter test catheter 1 or the probe 2 is now pulled from the urethra 26 at constant speed, for example using a catheter pulling device, the spatial course of the urethra 26 can be determined on the basis of the change in orientation of the orientation sensor 11, which is useful in particular in the diagnosis of a prolapsed bladder (FIG. 5(b)) in female patients. In the event of a prolapsed bladder, the normally straight urethra (FIG. 5(a)) has a kink (FIG. 5(b)). Here, the orientation sensor 11 is ideally arranged at the proximal end of the probe 2.

Whereas in laboratories or in doctors' surgeries corresponding sphincter test catheters are directly connected to stationary recording units 4, it is more useful for long-term monitoring to secure a mobile apparatus to the patient. Such a mobile apparatus comprises a battery and an electronic recording apparatus in which the measured data is checked and stored at predefinable time intervals. This mobile apparatus may comprise a transmitter by means of which the recorded data is forwarded to a receiver, which for example is connected to a stationary measuring and recording unit. Of course, however, the measured data may also be stored in a sufficiently large memory of the mobile apparatus on the patient and only wiped at the end of the measurement, when the patient returns for example to the doctor's surgery, the corresponding recorded information being compiled in a desired form from said data.

The solution according to the invention, in which the orientation sensor is placed intracorporeally, guarantees a reliable position determination, which is independent of the influence of the patient. Reliable values are thus obtained, and there is additionally the advantage that the patient merely carries one connection lead, which exits from the patient in the form of the catheter body 3 and is connected directly or indirectly to a recording unit 4. This recording unit 4 is preferably a mobile unit, which is attached directly to the patient himself/herself.

### LIST OF REFERENCE SIGNS

1. sphincter test catheter
2. probe
3. catheter body
4. recording unit
10. pressure sensor (AP)
11. orientation sensor (LS)
12. pH value sensor (pH)
13. additional pH value sensor
14. impedance sensor (Z)
5. electrical conductors
6. sockets
20. oesophagus
21. lower oesophageal sphincter
22. stomach
24. bladder
25. urethral sphincter
26. urethra

1. A sphincter test catheter for testing and monitoring the function of an intracorporeal sphincter comprising a flexible catheter body having a probe arranged at the proximal end thereof, in which probe at least one pressure sensor is provided, wherein the sphincter catheter can be brought into connection with a measuring and recording unit wherein, in order to monitor the bodily position of a patient, at least one orientation sensor is additionally provided in the probe in combination with either a pH value sensor and/or an impedance sensor.

2. The sphincter test catheter according to claim 1, wherein the orientation sensor is located in the probe in the region of the proximal end, and the pressure sensor is arranged first in the distal direction, followed by the pH value sensor.

3. The sphincter test catheter according to claim 1, wherein the pressure sensor is located in the probe in the region of the proximal end, and the orientation sensor is arranged first in the distal direction, followed by the impedance sensor.

4. The sphincter test catheter according to claim 2, wherein an additional pH value sensor is arranged between the orientation sensor and the pressure sensor.

5. The sphincter test catheter according to claim 1, wherein the probe is connected via the catheter body to a transmitter, which can be secured to the patient and which makes it possible to forward on the signals of the sensors, to a receiver of the measuring and recording unit.

6. The sphincter test catheter according to claim 2, wherein the distance between the pressure sensor and the orientation sensor is at least 2 cm and at most 8 cm.

7. The sphincter test catheter according to claim 2, wherein the distance between the pressure sensor and the pH value probe following in the distal direction is between 2 cm and 10 cm.

8. A measuring and recording unit for a sphincter test catheter according to claim 1, wherein the measuring and recording unit is configured in such a way that it determines and records the change in the spatial orientation of the orientation sensor in accordance with the time over which the catheter is removed from the urethra at a constant speed, for example with the aid of a catheter pulling device.

9. The measuring and recording unit according to claim 8, wherein said unit is configured to calculate the spatial course of the urethra from the time-dependent change of the spatial orientation of the orientation sensor.
